(19) United States
(12) Patent Application Publication
(10) Pub. No.: US 2025/0144443 A1
(43) Pub. Date: May 8, 2025

(54) Lighting Apparatus With Gamma Stimulation

(71) Applicant: Aleddra Inc., Renton, WA (US)

(72) Inventors: Chia-Yiu Maa, Bellevue, WA (US); Li-Jyuan Luo, Taipei City (TW); Chun-Te Yu, Bellevue, WA (US)

(21) Appl. No.: 18/613,079

(22) Filed: Mar. 21, 2024

Related U.S. Application Data
(63) Continuation-in-part of application No. 18/408,523, filed on Jan. 9, 2024.
Application No. 18/408,523 is a continuation-in-part of application No. 18/198,052, filed on May 16, 2023.
Application No. 18/198,052 is a continuation-in-part of application No. 18/101,569, filed on Jan. 25, 2023, now Pat. No. 12,127,314.
Application No. 18/101,569 is a continuation-in-part of application No. 17/981,123, filed on Nov. 4, 2022, now Pat. No. 12,048,078.
Application No. 17/981,123 is a continuation-in-part of application No. 17/509,877, filed on Oct. 25, 2021, now abandoned.
Application No. 17/509,877 is a continuation-in-part of application No. 17/148,277, filed on Jan. 13, 2021, now Pat. No. 11,191,863.
Application No. 17/148,277 is a continuation-in-part of application No. 17/094,567, filed on Nov. 10, 2020, now Pat. No. 11,103,612.
Application No. 17/094,567 is a continuation-in-part of application No. 16/180,416, filed on Nov. 5, 2018, now Pat. No. 10,874,762.

(51) Int. Cl. A61N 5/06

(52) U.S. Cl. CPC A61N 5/0622; A61N 2005/0626; A61N 2005/0653

(57) ABSTRACT

A lighting apparatus comprises a controller and a light source. The controller is configured to operate the light source according to a periodical waveform at a first frequency (F1) between 20 Hz and 45 Hz, and the light output of the lighting apparatus appears flicker-free (free of flicker) to a subject. The periodical waveform is decomposable into a first periodical baseline waveform at a second frequency and a second periodical baseline waveform at a third frequency such that the first frequency equal to the difference of the third frequency and the second frequency.

### BACKGROUND

The present disclosure is a continuation-in-part (CIP) of U.S. patent application Ser. No. 18/408,523, filed 9 Jan. 2024. Content of aforementioned application is herein incorporated by reference in its entirety.

### TECHNICAL FIELD

The present disclosure pertains to the field of lighting apparatus and, more specifically, proposes lighting apparatus with gamma stimulation.

### DESCRIPTION OF RELATED ART

It has been discovered that by flickering a light at a frequency between 35 Hz to 45 Hz or generating a sound at a similar frequency has the effect of stimulating the cells in certain region of the brain, resulting in using a flicking light or a sound at such a frequency for treating Alzheimer's disease. However, turning on and off a light source at a frequency between 35 Hz to 45 Hz can create visual discomfort to the eyes of a subject. Different approaches have been introduced to overcome this visual discomfort under 40 Hz flickering light.

One of the approaches in U.S. patent application Ser. No. 18/408,523 introduces the use of a controller and two light sources such that the controller would operate these two light sources at two different frequencies resulting a superimposed light operating at a third frequency equal to the difference of these two frequencies. The operating frequency of the first light source is ≥50 Hz and the operating frequency of the second light source is greater than the operating frequency of the first light source by at least 30 Hz but no more than 65 Hz.

The present disclosure proposes a lighting apparatus for generating gamma visual stimulation between 20 Hz and 45 Hz by using a smart controller and only one light source such that the smart controller can operate the one light source according to a superimposed waveform for creating the same gamma stimulation waveform that was created previously by using two light sources each operating at a different frequency.

### SUMMARY

One of the approaches in U.S. patent application Ser. No. 18/408,523 introduces the use of a controller and two light sources such that the controller would operate these two light sources at two different frequencies resulting a superimposed light operating according to a third frequency equal to the difference of these two frequencies. FIG. 1 demonstrates this approach where the first light source operates according to the first waveform at 8 Hz and the second light source operates according to the second waveform at 12 Hz, resulting in a superimposed light having a third frequency at 4 Hz=12 Hz−8 Hz. Using a smart controller such as a microprocessor, it is feasible to generate a periodical waveform same as the superimposed waveform in FIG. 1, by either superimposing the first waveform and the second waveform internally in the smart controller or by storing the superimposed waveform data internally in the smart controller. Such smart controller can then operate a single light source according to this superimposed waveform directly, resulting in the same superimposed light that required the use of two light sources each operating at a different frequency previously.

In one aspect, the lighting apparatus comprises a controller and a light source. The controller is configured to operate the light source according to a periodical waveform at a first frequency (F1) between 20 Hz and 45 Hz. The periodical waveform is decomposable into a first periodical baseline waveform at a second frequency (F2) and a second periodical baseline waveform at a third frequency (F3) such that F1=F3-F2. Using FIG. 1 as example (by scaling the frequencies of three waveforms 10 times), the controller operates the light source according to the superimposed waveform at 40 Hz, and the superimposed waveform can be decomposed into a first periodical baseline waveform operating at 80 Hz (as shown by the first waveform in FIG. 1 scaling up 10 times) and a second periodical baseline waveform operating at 120 Hz (as shown by the second waveform in FIG. 1 scaling up 10 times). Moreover, the light output of the lighting apparatus appears flicker-free (free of flicker) to the eyes of a subject.

FIG. 1 to FIG. 3 show examples of how each red periodical waveform can be formed by superimposing the corresponding first periodical waveform and the corresponding second periodical waveform. However, the clamped superimposed waveform in FIG. 4 is produced by clamping the output of the superimposed waveform in FIGS. 1 to 10. Such clamped, superimposed waveform is hard to construct by superimposing the first waveform and the second waveform shown in FIG. 4. Nonetheless, a smart controller may still operate the light source directly according to its in-store data of the clamped, superimposed waveform shown in FIG. 4.

It is to be noted that while the periodical waveform of the light apparatus may be formed by superimposing the first periodical baseline waveform and the second periodical baseline, it doesn't mean the controller is necessarily performing an act of superimposition of the first periodical baseline waveform and the second periodical baseline, nor does it rule out such implementation. For example, it may be feasible to store the data of such periodical waveform in the controller, without taking any action on superimposing of two baseline periodical waveforms, and the controller operate the light source according to the stored data of such periodical waveform.

In some embodiments, the first periodical baseline waveform and the second periodical baseline waveform have the same waveform style (e.g. sinusoidal, rectangle, square, triangle, trapezoidal, etc.), but differ in frequency. FIG. 1 to FIG. 3 show examples of sinusoidal waveforms and trapezoidal waveforms.

In some embodiments, the periodical waveform has more than one peak within a full cycle. The periodical waveforms shown in FIG. 1 to FIG. 4 all have more than one peak within a full cycle. More than one peak within a full cycle gives the eyes of a subject the illusion that the frequency of the periodical waveform is higher than it really is. For example, if there are two peaks within a full cycle of the periodical waveform (e.g., operating at 40 Hz), then the eyes of the subject may have the illusion that the periodical waveform is operating at twice its true frequency (e.g., 80 Hz), and thus the eyes may not perceive the 40 Hz flickering. Similarly, if there are three peaks within a full cycle of the periodical waveform (e.g., operating at 40 Hz), then the eyes of the subject may have the illusion that the periodical waveform is operating at three times its true frequency (e.g., 120 Hz), and thus the eyes may not perceive the 40 Hz flickering.

In some embodiments, F1 frequency is chosen to be 40 Hz since it is known to trigger the best gamma stimulation effect.

In some embodiments, F2 frequency is greater than 50 Hz.

In some embodiments, F2 frequency is 80 Hz. and F3 frequency is 120 Hz.

In some embodiments, the light source comprises a light emitting diode (LED) or organic LED (OLED).

### BRIEF DESCRIPTION OF THE DRAWINGS

The accompanying drawings are included to aid further understanding of the present disclosure and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1 schematically depicts the superimposing of two sinusoidal waveforms at F1=8 Hz and F2=12 Hz.

FIG. 2 schematically depicts the superimposing of two trapezoidal waveforms at F1=8 Hz and F2=12 Hz.

FIG. 3 schematically depicts the superimposing of two more trapezoidal waveforms at F1=8 Hz and F2=12 Hz with longer On-state duration.

FIG. 4 schematically depicts a clamped superimposed waveform.

FIG. 5 schematically depicts an embodiment of the present disclosure using one light source.

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of lighting apparatuses having different form factors.

A lighting apparatus comprises a controller and a light source. The controller is configured to operate the light source according to a periodical waveform at a first frequency (F1) between 20 Hz and 45 Hz, and the light output of the lighting apparatus appears flicker-free (free of flicker) to a subject. The periodical waveform is decomposable into a first periodical baseline waveform at a second frequency and a second periodical baseline waveform at a third frequency such that the first frequency equal to the difference of the third frequency and the second frequency.

### Example Implementations

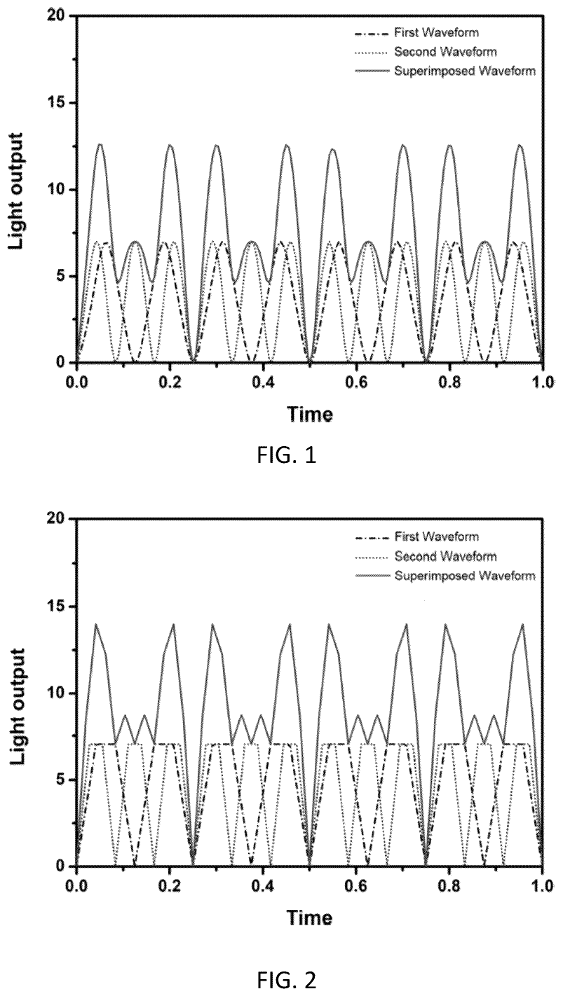

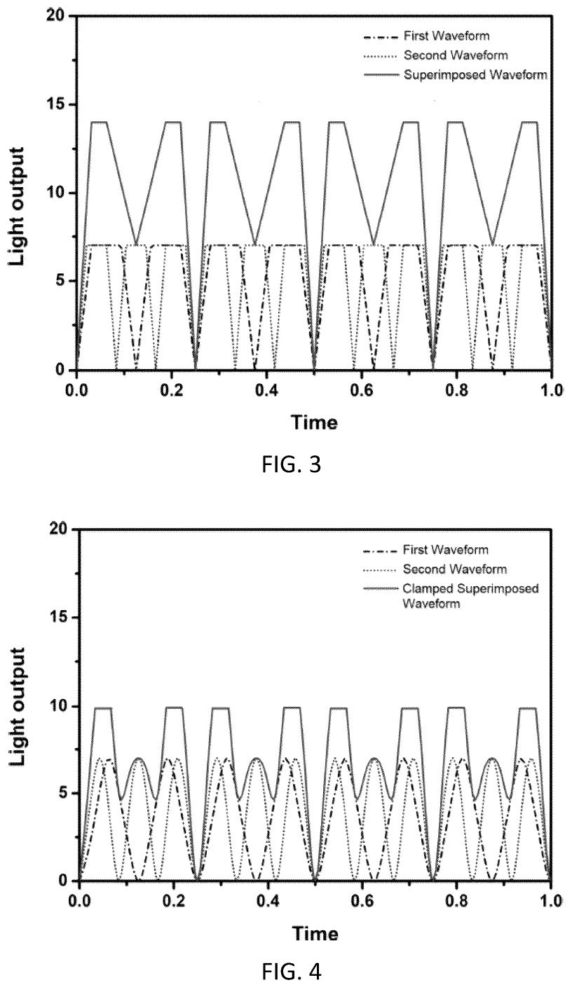

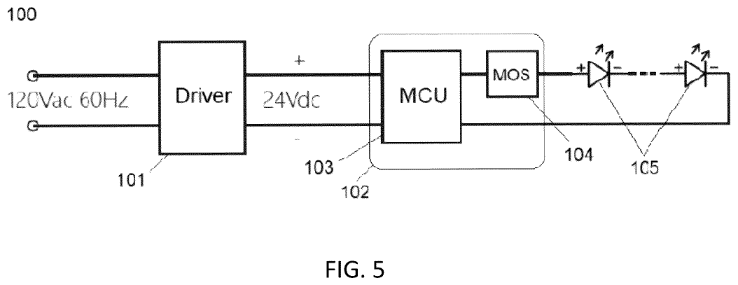

FIG. 5 shows an embodiment of the lighting apparatus of the present disclosure 100. It comprises a driver 101, an MCU 103, an MOS 104, and one light source 105 comprising multiple LEDs. The driver 101, the MCU 103, and the MOS 104 together form the controller of the lighting apparatus. The MCU 103 and the MOS 104 determine how the light source 105 would operate. The MCU 103 may superimpose internally the first baseline waveform and the second baseline waveform shown in FIG. 1 to FIG. 3 for creating the superimposed waveform and then instruct the MOS 104 to operate the light source 105 according to the superimposed waveform. Or alternatively, the MCU 103 may have the data of a periodical waveform as shown by the red waveform in FIG. 1 to FIG. 4 locally (scaled to 40 Hz), and thus instruct the MOS 104 to operate the light source 105 according to the stored periodical waveform, without doing any superimposition operation of two periodical baseline waveforms.

### Additional and Alternative Implementation Notes

Although the techniques have been described in language specific to certain applications, it is to be understood that the appended claims are not necessarily limited to the specific features or applications described herein. Rather, the specific features and examples are disclosed as non-limiting exemplary forms of implementing such techniques.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. A lighting apparatus, comprising:
a controller; and
a light source,
wherein:
the controller is configured to operate the light source according to a periodical waveform at a first frequency (F1) between 20 Hz and 45 Hz,
the periodical waveform is decomposable into a first periodical baseline waveform at a second frequency (F2) and a second periodical baseline waveform at a third frequency (F3) such that F1=F3-F2, and
a light output of the lighting apparatus appears flicker-free (free of flicker) to eyes of a subject.

2. The lighting apparatus of claim 1, wherein the first periodical baseline waveform and the second periodical baseline waveform have a same waveform style but differ in frequency.

3. The lighting apparatus of claim 1, wherein the periodical waveform has more than one peak within a full cycle.

4. The lighting apparatus of claim 1, wherein the F1 frequency is 40 Hz.

5. The lighting apparatus of claim 1, wherein the F2 frequency is greater than 50 Hz.

6. The lighting apparatus of claim 5, wherein the F2 frequency is 80 Hz. and the F3 frequency is 120 Hz.

7. The lighting apparatus of claim 1, wherein the light source comprises a light emitting diode (LED) or organic LED (OLED).
